(19) United States
(12) Patent Application Publication
(10) Pub. No.: US 2026/0335359 A1
(43) Pub. Date: Oct. 29, 2026

(54) SUPERPARAMAGNETIC IRON OXIDE NANOPARTICLES FOR IMMUNOTHERAPY

(71) Applicants: SEOUL NATIONAL UNIVERSITY R&DB FOUNDATION, Seoul (KR); BRAIN & BEYONDS CO., LTD., Seoul (KR)

(72) Inventors: Sun Ha PAEK, Seoul (KR); Soon Beom PARK, Seoul (KR)

(73) Assignees: SEOUL NATIONAL UNIVERSITY R&DB FOUNDATION, Seoul (KR); BRAIN & BEYONDS CO., LTD., Seoul (KR)

(21) Appl. No.: 19/104,001

(22) PCT Filed: Aug. 11, 2023
(86) PCT No.: PCT/KR2023/011974

§ 371 Date: Feb. 14, 2025

(30) Foreign Application Priority Data
Aug. 17, 2022 (KR) 10-2022-0102427

(51) Int. Cl. A61K 33/32; A61K 9/14; A61K 9/50; A61P 35/00; A61P 37/04

(52) U.S. Cl. CPC A61K 33/32; A61K 9/14; A61K 9/5031; A61P 35/00; A61P 37/04

(57) ABSTRACT

The present invention relates to a composition for cancer immunotherapy, comprising superparamagnetic nanoparticles as an active ingredient, a composition for treating immune diseases, a composition for enhancing immunity, a composition for suppressing a decrease in immune function or enhancing immune function, the composition being administered in combination with a chemotherapeutic anticancer drug, a composition for suppressing a decrease in immune function, caused by anticancer treatment or enhancing immune function, and a composition for ameliorating, treating or preventing decreased immune responses in individuals with cancer. The superparamagnetic nanoparticles according to the present invention have magnetically-induced heating characteristics and high biocompatibility, thus being applicable through in vivo injection, and can induce an increase in immune cells, thus being effectively used in the treatment of cancer and immune diseases.

### TECHNICAL FIELD

The present disclosure relates to a composition for cancer immunotherapy, a composition for treating immune diseases, a composition for enhancing immunity, a composition for suppressing a decrease in immune function or enhancing immune function, administered in combination with a chemotherapeutic anticancer drug, a composition for suppressing a decrease in immune function, caused by anticancer treatment or enhancing immune function, and a composition for ameliorating, treating or preventing a decrease in immune responses in individuals with cancer, with the composition comprising superparamagnetic nanoparticles as an active ingredient.

### BACKGROUND ART

In modern times, many diseases have become easily treatable, and there are almost no diseases that are incurable. However, cancer, unlike other diseases, requires very difficult and complex treatments, and these complex treatments are also not fully effective. The main methods currently used for cancer treatment include surgery, radiation therapy, and chemotherapy. When cancer develops, surgery will be performed to remove the cancer, but if the cancer has not spread far and is only localized, a complete cure can be achieved through surgery. However, 70% or more of patients tend to have cancer that has metastasized, so adjuvant therapies are performed concurrently. Radiation therapy, one of the adjuvant therapies, is a treatment that uses high-energy radiation to kill cancer cells. When radiation is applied to cancer, it does not kill the cancer cells immediately, but it destroys the ability of the cancer cells to proliferate, preventing new cancer cells from being created and dividing any longer. However, this method has a side effect that affects not only cancer cells but also normal cells. Chemotherapy is an adjuvant therapy that uses drugs to kill cancer cells after surgery, and is performed to kill cancer cells that cannot be seen. However, chemotherapy can also cause side effects such as vomiting, diarrhea, and hair loss.

To minimize these side effects, immunotherapy methods have recently emerged. In addition, as mentioned above, the metastasis rate of cancer exceeds 70% of all patients, SO treating metastatic cancer can be considered essential for a complete cure, and immunotherapy is a very effective treatment method in this regard.

Immunotherapy is a method of treating diseases by utilizing an immune response in the patient's body. However, most of the currently used treatments for autoimmune diseases tend to prescribe immunosuppressive drugs that suppress excessive inflammatory responses, rather than treatments that suppress the underlying cause of the disease, and biological agents developed through recent research have also failed to be fundamental treatments for a complete cure.

On the other hand, magnetic nanoparticles have magnetic-induced heating characteristics when exposed to an alternating current magnetic field, and a hyperthermia effect obtained by specific exposure to a desired area may be applied to various treatments. In recent years, a new type of treatment method that combines hyperthermia therapy with chemotherapy and radiation therapy has gained attention.

Against this background, the present inventors have conducted intensive research on a medium that can enhance immune function to overcome side effects caused by cancer, immune diseases, or anticancer chemotherapy, and as a result, confirmed that immune function may be enhanced by superparamagnetic nanoparticles Mn_{0.5}Zn_{0.5}Fe₂O₄, thereby completing the present disclosure.

### TECHNICAL PROBLEM

The present disclosure aims to solve the aforementioned problems and other problems related thereto.

An exemplary object of the present disclosure is to provide a composition for cancer immunotherapy, comprising a superparamagnetic nanoparticle represented by the following Formula 1 as an active ingredient:

[Formula 1]

KₓL_{y}Fe₂₍₃₎O₄ (0≤x≤3, 0≤y≤3)

wherein K or L is a monovalent to trivalent cation metal including lithium (Li), sodium (Na), magnesium (Mg), calcium (Ca), manganese (Mn), zinc (Zn), aluminum (Al), or gallium (Ga).

Another exemplary object of the present disclosure is to provide a composition for cancer immunotherapy, comprising a superparamagnetic nanoparticle represented by the following Formula 2 as an active ingredient:

[Formula 2]

KₓL_{y}M_{z}Fe₂₍₃₎O₄ (0≤x≤3, 0≤y≤3, 0≤z≤3)

wherein K, L, or M is a monovalent to trivalent cation metal including lithium sodium (Na), magnesium (Mg), calcium (Ca), manganese (Mn), zinc (Zn), aluminum (Al), or gallium (Ga).

Another exemplary object of the present disclosure is to provide a composition for treating an immune disease, comprising a superparamagnetic nanoparticle represented by the Formula 1 or Formula 2 as an active ingredient.

Another exemplary object of the present disclosure is to provide a composition for enhancing immunity, comprising a superparamagnetic nanoparticle represented by the Formula 1 or Formula 2 as an active ingredient.

Another exemplary object of the present disclosure is to provide a composition for suppressing a decrease in immune function or enhancing immune function, administered in combination with a chemotherapeutic anticancer drug, comprising a superparamagnetic nanoparticle represented by the Formula 1 or Formula 2 as an active ingredient.

Another exemplary object of the present disclosure is to provide a composition for suppressing a decrease in immune function, caused by anticancer treatment or enhancing immune function, comprising a superparamagnetic nanoparticle represented by the Formula 1 or Formula 2 as an active ingredient.

Another exemplary object of the present disclosure is to provide a composition for ameliorating, treating, or preventing a decrease in immune responses in individuals with cancer, comprising a superparamagnetic nanoparticle represented by the Formula 1 or Formula 2 as an active ingredient.

The technical problems to be solved in accordance with the technical ideas of the present disclosure disclosed herein are not limited to addressing those mentioned above, and other problems not mentioned will be clearly understood by those skilled in the art from the following description.

### TECHNICAL SOLUTION

This will be specifically described as follows. Meanwhile, each of the descriptions and embodiments disclosed in the present application may also be applied to each other descriptions and embodiments. That is, all combinations of various elements disclosed in the present application fall within the scope of the present application. In addition, the scope of the present application cannot be considered limited by the specific description set forth below.

An aspect of the present disclosure provides a composition for cancer immunotherapy, comprising a superparamagnetic nanoparticle represented by the following Formula 1 as an active ingredient:

[Formula 1]

KₓL_{y}Fe₂₍₃₎O₄ (0≤x≤3, 0≤y≤3)

wherein K or L is a monovalent to trivalent cation metal including lithium (Li), sodium (Na), magnesium (Mg), calcium (Ca), manganese (Mn), zinc (Zn), aluminum (Al), or gallium (Ga).

Specifically, x and y may be numbers between 0 and 3, for example, 0.1, 0.2, 0.3, 0.4, 0.5, 0.6, 0.7, 0.8, 0.9, 1.0, 1.1, 1. 2, 1. 3, 1.4, 1.5, 1.6, 1. 7, 1.8, 1.9, 2.0, 2.1, 2.2, 2.3, 2. 4, 2.5, 2.6, 2.7, 2.8, 2.9, or 3.0.

More specifically, the superparamagnetic nanoparticles may be MnₓZn₁₋ₓFe₂O₄, and more specifically, the superparamagnetic nanoparticles may be Mn_{0.5}Zn_{0.5}Fe₂O₄.

Another aspect of the present disclosure provides a composition for cancer immunotherapy, comprising a superparamagnetic nanoparticle represented by the following Formula 2 as an active ingredient:

[Formula 2]

KₓL_{y}M_{z}Fe₂₍₃₎O₄ (0≤x≤3, 0≤y≤3, 0≤z≤3)

wherein K, L, or M is a monovalent to trivalent cation metal including lithium (Li), sodium (Na), magnesium (Mg), calcium (Ca), manganese (Mn), zinc (Zn), aluminum (Al), or gallium (Ga).

Specifically, x, y, and z may be numbers between 0 and 3, for example, 0.1, 0.2, 0.3, 0.4, 0.5, 0.6, 0.7, 0.8, 0.9, 1.0, 1. 1, 1.2, 1. 3, 1.4, 1.5, 1.6, 1. 7, 1.8, 1.9, 2.0, 2.1, 2.2, 2.3, 2.4, 2.5, 2.6, 2.7, 2.8, 2.9, or 3.0.

In the present disclosure, the superparamagnetic nanoparticles may be coated with a biocompatible polymer.

A biocompatible polymeric material useful for the human body that may be used in the present disclosure may be a polymer that are readily soluble in various solvents, for example, one or more selected from the group consisting of polyethyleneglycol (PEG), poly(lactide-co-glycolide) (PLGA), poly(DL-lactide-co-glycolide) (PDLGA), poly(hydroxybutyrate), and polycaprolactone (PCL), and specifically, polyethyleneglycol.

The term “cancer immunotherapy” as used herein refers to a method of treating cancer by using an immune response in the patient's body. This method involves activating cancer-specific immune cells and then causing the activated immune cells to specifically attack cancer in the body.

In the present disclosure, the cancer immunotherapy may be achieved by injecting superparamagnetic nanoparticles into the subject's body, and specifically, by an increase in immune cells by the superparamagnetic nanoparticles, wherein the immune cells may be one or more selected from the group consisting of, but are not limited to, macrophages, natural killer cells, and T cells.

The composition for cancer immunotherapy comprising superparamagnetic nanoparticles according to the present disclosure as an active ingredient may achieve therapeutic efficacy by direct injection into the body.

The composition for cancer immunotherapy comprising superparamagnetic nanoparticles according to the present disclosure may Selectively and uniformly generate high temperatures because the magnetic nanoparticles dispersed in the target organ may continuously react with an external magnetic field when applied. This treatment method may be performed as alone, in combination with a conventional treatment method, or as an adjuvant treatment method.

Specifically, in an embodiment of the present disclosure, it was confirmed that when superparamagnetic nanoparticles Mn_{0.5}Zn_{0.5}Fe₂O₄ were injected into a fibrosarcoma mouse model and a glioblastoma mouse model, respectively, and an alternating current magnetic field was applied, an increase in immune cells such as macrophages, natural killer cells, and T cells occurred around cancer cells, and the tumor size decreased.

The composition for cancer immunotherapy of the present disclosure is typically provided as a pharmaceutical composition. Accordingly, the composition for cancer immunotherapy of the present disclosure comprises a pharmaceutically acceptable carrier. The pharmaceutically acceptable carriers are those typically used during formulation. Examples thereof include, but are not limited to, lactose, dextrose, sucrose, sorbitol, mannitol, starch, acacia gum, calcium phosphate, alginate, gelatin, calcium silicate, microcrystalline cellulose, polyvinylpyrrolidone, cellulose, water, syrup, methyl cellulose, methylhydroxybenzoate, propylhydroxybenzoate, talc, magnesium stearate, or mineral oil, etc. Suitable pharmaceutically acceptable carriers and formulations are described in detail in Remington's Pharmaceutical Sciences, 19th ed., 1995.

The composition for cancer immunotherapy of the present disclosure is preferably administered parenterally. In the case of parenteral administration, it can be administered by intravenous injection, subcutaneous injection, intramuscular injection, intraperitoneal injection, or intralesional injection, etc. Suitable dosages of the compositions of the present disclosure may be prescribed in various ways depending on factors such as the formulation method, administration method, patient's age, weight, sex, pathological condition, food, administration time, administration route, excretion rate, and response sensitivity. The composition for cancer immunotherapy of the present disclosure comprises a therapeutically effective amount of superparamagnetic nanoparticles exhibiting a magnetic-induced heating effect. The term “therapeutically effective amount” refers to an amount sufficient to treat the target disease, and is generally 0.0001 to 100 mg/kg.

The pharmaceutical composition of the present disclosure may be prepared in a unit dosage form by formulating it using a pharmaceutically acceptable carriers and/or excipient, or may be prepared by introducing it into multi-dose containers, according to a method readily practicable by those skilled in the art to which the present disclosure belongs. Here, the formulation may be in the form of an oil or a solution, suspension, or emulsion in an aqueous medium, or in the form of an extract, powder, granule, tablet, or capsule, and may further comprise a dispersant or a stabilizer.

In the present disclosure, the cancer may be, but is not limited to, fibrosarcoma, brain cancer, lung cancer, colorectal cancer, liver cancer, breast cancer, stomach cancer, ovarian cancer, skin cancer, pancreatic cancer, prostate cancer, kidney cancer, or thyroid cancer.

Another aspect of the present disclosure provides a composition for treating an immune disease comprising a superparamagnetic nanoparticle represented by the Formula 1 or Formula 2 as an active ingredient.

The superparamagnetic nanoparticle is as described above.

In the present disclosure, the treatment of an immune disease may be achieved by injecting superparamagnetic nanoparticles into the subject's body, and specifically, by an increase in immune cells by the superparamagnetic nanoparticles, wherein the immune cells may be one or more selected from the group consisting of, but are not limited to, macrophages, natural killer cells, and T cells.

The composition for treating an immune disease comprising superparamagnetic nanoparticles according to the present disclosure as an active ingredient may achieve therapeutic efficacy by direct injection into the body.

The composition for treating an immune disease comprising superparamagnetic nanoparticles according to the present disclosure may selectively and uniformly generate high temperatures because the magnetic nanoparticles dispersed in the target organ may continuously react with an external magnetic field when applied. This treatment method may be performed as alone, in combination with a conventional treatment method, or as an adjuvant treatment method.

The composition for treating an immune disease of the present disclosure is typically provided as a pharmaceutical composition. The acceptable carrier, administration and preparation of the pharmaceutical composition are as described above.

The immune diseases may include a disease related to a weakened immune system of human body or a disease related to an overactive immune system of human body, and may specifically include an immunodeficiency disease, an infectious disease, or an autoimmune disease.

The diseases related to a weakened immune system of human body may typically include, but are not limited to, congenital immunodeficiency diseases, acquired immunodeficiency diseases, bacterial and viral infectious diseases (e.g., various bacterial infectious diseases, intractable viral infections including COVID-19, SARS, MERS, etc.). In the case of diseases related to limited immune system of human body, the temperature may be appropriately controlled by applying an alternating current magnetic field to superparamagnetic nanoparticles to prevent excessive heat generation, thereby activating immune cells with impaired function.

The diseases related to an overactive immune system of human body may include, but are not limited to, an autoimmune disease. In the case of such diseases related to an overactive immune system of human body, superparamagnetic nanoparticles subjected to an alternating current magnetic field may generate heat to destroy overactive immune cells, thereby treating the disease.

In the present disclosure, the autoimmune disease may be selected from the group consisting of lupus (systemic lupus erythematosus), rheumatoid arthritis, progressive systemic sclerosis (scleroderma), atopic dermatitis, alopecia areata, psoriasis, pemphigus, asthma, aphthous stomatitis, chronic thyroiditis, inflammatory enteritis, Behcet's disease, Crohn's disease, dermatomyositis, polymyositis, multiple sclerosis, autoimmune hemolytic anemia, autoimmune encephalomyelitis, myasthenia gravis, Grave's disease, polyarteritis nodosa, ankylosing spondylitis, fibromyalgia syndrome, and temporal arteritis.

Another aspect of the present disclosure provides a composition for enhancing immunity, comprising a superparamagnetic nanoparticle represented by the Formula 1 or Formula 2 an active ingredient.

The superparamagnetic nanoparticle is as described above.

As used herein, the term “immune enhancement” refers to a function that promotes an immune response to an antigen non-specifically during the initial activation of immune cells, or a function that enhances immunity by increasing the activity of immune system cells, and an immune-enhancing effect may be achieved by stimulating the immune response by increasing the activity of immune cells. For example, macrophages play a major role in the immune response. The main role of macrophages is phagocytosis, which involves absorbing microorganisms and other pyrogenic particles, and also stimulating the immune response by secreting a number of cytokines, such as tumor necrosis factor-α (TNF-α), interleukin-1β (IL-1β), and interleukin-12 (IL-12), and cytotoxic and inflammatory substances, such as nitric oxide (NO) (Wolf et al., 1994; Lee and Hong, 2011; Murray and Wynn, 2011). Therefore, increasing macrophage activity may be one means of enhancing immunity.

In the present disclosure, the immune enhancement may be achieved by injecting superparamagnetic nanoparticles into the body of a subject, and specifically, by an increase in immune cells by the superparamagnetic nanoparticles, wherein the immune cells may be one or more selected from the group consisting of, but are not limited to, macrophages, natural killer cells, and T cells.

The composition for enhancing immunity comprising superparamagnetic nanoparticles according to the present disclosure as an active ingredient may achieve therapeutic efficacy by direct injection into the body.

The composition for enhancing immunity comprising superparamagnetic nanoparticles according to the present disclosure may Selectively and uniformly generate high temperatures because the magnetic nanoparticles dispersed in the target organ may continuously react with an external magnetic field when applied. This treatment method may be performed as alone, in combination with a conventional treatment method, or as an adjuvant treatment method.

The composition for enhancing immunity of the present disclosure is typically provided as a pharmaceutical composition. The acceptable carrier, administration and preparation of the pharmaceutical composition are as described above.

Another aspect of the present disclosure provides a composition for suppressing a decrease in immune function or enhancing immune function, administered in combination with chemotherapeutic anticancer drugs, comprising a superparamagnetic nanoparticle represented by the Formula 1 or Formula 2 as an active ingredient.

As used herein, the term “combination administration” may be achieved by administering the individual components of a therapeutic regimen simultaneously, sequentially, or separately. The combination therapeutic effects may be achieved by administering two or more drugs or compositions simultaneously or sequentially, or alternately at regular or undetermined intervals. A combination therapy may be defined, without limitation, as one in which the efficacy measured, for example, through the degree of response, rate of response, the period until disease progression, or the survival period, is therapeutically superior to that which would be obtained by administering one or the remaining components of the combination therapy at a regular dose, while providing a synergistic effect.

When the superparamagnetic nanoparticles of the present disclosure are administered in combination with an anticancer drug, the increased number of immune cells may effectively kill cancer cells, thus exhibiting a synergistic anticancer treatment.

In the present disclosure, the anticancer drug may be, for example, but is not limited to, eribulin, carboplatin, cisplatin, Halaven, 5-fluorouracil (5-FU), Glivec, vincristine, vinblastine, vinorelbine, paclitaxel, docetaxel, etoposide, topotecan, irinotecan, dactinomycin, doxorubicin, daunorubicin, valrubicin, flutamide, gemcitabine, mitomycin, or bleomycin.

In the present disclosure, the suppression of a decrease in immune function or the enhancement of immune function may be achieved by injecting superparamagnetic nanoparticles into the subject's body, and specifically, by an increase in immune cells by the superparamagnetic nanoparticles, wherein the immune cells may be one or more selected from the group consisting of, but are not limited to, macrophages, natural killer cells, and T cells.

A composition for suppressing a decrease in immune function or enhancing immune function, administered in combination with a chemotherapeutic anticancer drug, comprising superparamagnetic nanoparticles according to the present disclosure as an active ingredient, may achieve therapeutic efficacy by direct injection into the body.

The composition for suppressing a decrease in immune function or enhancing immune function, administered in combination with a chemotherapeutic anticancer drug, comprising superparamagnetic nanoparticles according to the present disclosure as an active ingredient, may selectively and uniformly generate high temperatures because the magnetic nanoparticles dispersed in the target organ may continuously react with an external magnetic field when applied. This treatment method may be performed as alone, in combination with a conventional treatment method, or as an adjuvant treatment method.

The composition for suppressing a decrease in immune function or enhancing immune function, administered in combination with a chemotherapeutic anticancer drug, comprising superparamagnetic nanoparticles according to the present disclosure as an active ingredient, is typically provided as a pharmaceutical composition. The acceptable carrier, administration and preparation of the pharmaceutical composition are as described above.

Another aspect of the present disclosure provides a composition for suppressing a decrease in immune function, caused by anticancer treatment or enhancing immune function, comprising a superparamagnetic nanoparticle represented by the Formula 1 or Formula 2 as an active ingredient.

The superparamagnetic nanoparticle is as described above.

In the present disclosure, the anticancer treatment may be, for example, but is not limited to, chemotherapy in which an anticancer drug is administered, or radiation therapy.

Conventional anticancer treatments cause fatal damage not only to cancer cells but also to normal cells, resulting in a decrease in immune function and side effects such as hair loss, diarrhea, gastrointestinal disorders, liver, and kidney toxicity, etc., and the decrease in immune function refers to a decrease in immune cell number and a decrease in immune cell activity. Therefore, when the superparamagnetic nanoparticles of the present disclosure are administered to an individual undergoing anticancer treatment, the decrease in immune function, caused by anticancer treatment may be suppressed or the immune function may be effectively enhanced.

In the present disclosure, the suppression of a decrease in immune function or the enhancement of immune function may be achieved by injecting superparamagnetic nanoparticles into the subject's body, and specifically, by an increase in immune cells by the superparamagnetic nanoparticles, wherein the immune cells may be one or more selected from the group consisting of, but are not limited to, macrophages, natural killer cells, and T cells.

The composition for suppressing a decrease in immune function, caused by anticancer treatment or enhancing immune function, comprising superparamagnetic nanoparticles according to the present disclosure as an active ingredient, is typically provided as a pharmaceutical composition. The acceptable carrier, administration and preparation of the pharmaceutical composition are as described above.

Another aspect of the present disclosure provides a composition for ameliorating, treating, or preventing a decrease in immune responses in individuals with cancer, comprising a superparamagnetic nanoparticle represented by the Formula 1 or Formula 2 as an active ingredient.

The superparamagnetic nanoparticle is as described above.

It is known that cancer cells suppress the immune function of the human body, for example, by stimulating immune checkpoint-related proteins and attenuate the activity of immune cells. Therefore, when the superparamagnetic nanoparticles of the present disclosure are administered to an individual with cancer, the immune cells are increased, and thus the decrease in immune function caused by the cancer cells may be suppressed or the immune function may be effectively enhanced.

In the present disclosure, the suppression of a decrease in immune function or the enhancement of immune function may be achieved by injecting superparamagnetic nanoparticles into the subject's body, and specifically, by an increase in immune cells by the superparamagnetic nanoparticles, wherein the immune cells may be one or more selected from the group consisting of, but are not limited to, macrophages, natural killer cells, and T cells.

The composition for ameliorating, treating, or preventing a decrease in immune responses in individuals with cancer, comprising superparamagnetic nanoparticles according to the present disclosure as an active ingredient, is typically provided as a pharmaceutical composition. The acceptable carrier, administration and preparation of the pharmaceutical composition are as described above.

In the present disclosure, for example, the cancer may be, but is not limited to, fibrosarcoma, brain cancer, lung cancer, colorectal cancer, liver cancer, breast cancer, stomach cancer, ovarian cancer, skin cancer, pancreatic cancer, prostate cancer, kidney cancer, or thyroid cancer.

Another exemplary object of the present disclosure is to provide a method for preparing a superparamagnetic nanoparticle comprising:
(a) mixing Fe (III) acetylacetonate, Mn (II) acetate tetrahydrate, Zn (II) acetate dihydrate, 1,2-hexadecanediol, oleic acid, oleylamine, and benzyl ether in a vessel;
(b) heating the vessel to 270 to 300 °C, followed by cooling to 250 to 280 °C;
(c) heating the vessel to 290 to 310 °C after the cooling, and maintaining the temperature; and
(d) lowering the temperature to 70 to 90 °C after the maintaining.

In the present disclosure, the heating in step (b) may be performed at a rate of 12 to 13 °C/min for 10 to 30 minutes, and specifically at a rate of 12 to 13 °C/min for 20 minutes.

In the present disclosure, the cooling in step (b) may be performed for 30 to 120 minutes, specifically for 30 to 90 minutes, and more specifically for 30 to 60 minutes.

In the present disclosure, the heating in step (c) may be performed at a rate of 2 to 3 °C/min for 5 to 20 minutes, and specifically for 5 to 10 minutes.

In the present disclosure, the maintaining in step (c) may be performed for 10 to 60 minutes, specifically for 20 to 50 minutes, more specifically for 30 to 50 minutes, even more specifically for 40 to 50 minutes.

Another aspect of the present disclosure provides a method for cancer immunotherapy comprising: administering to a subject (including a mammal such as a human, an animal, or a mouse) a composition comprising a superparamagnetic nanoparticle as an active ingredient.

Another aspect of the present disclosure provides a use for cancer immunotherapy of a superparamagnetic nanoparticle.

### Advantageous Effects

The superparamagnetic nanoparticles according to the present disclosure have magnetically-induced heating characteristics and high biocompatibility, thus being applicable through in vivo injection, and can induce an increase in immune cells, thus being effectively used in the treatment of cancer and immune diseases.

### DESCRIPTION OF DRAWINGS

FIG. 1 shows the thermal characteristic curves of superparamagnetic nanoparticles Mn_{0.5}Zn_{0.5}Fe₂O₄ prepared by the preparation method of the present disclosure and the conventional method, respectively (a: conventional method, b: method of Example 1-1).

FIG. 2 shows the yields by magnetic-inducted heating characteristics of superparamagnetic nanoparticles Mn_{0.5}Zn_{0.5}Fe₂O₄ prepared by the preparation method of the present disclosure and the conventional method, respectively. (a: conventional method, b: method of Example 1-1).

FIG. 3 is an AC magnetic field generation system for magnetically inducing heating in the superparamagnetic nanoparticles of the present disclosure.

FIG. 4 is a graph showing the magnetic-induced heating characteristics of superparamagnetic nanoparticles Mn_{0.5}Zn_{0.5}Fe₂O₄ synthesized by Example of the present disclosure.

FIG. 5 is the results of observing, with the naked eye, the change in tumor size over time in an FSaLL mouse tumor model as a control group and an experimental group to which superparamagnetic nanoparticles Mn_{0.5}Zn_{0.5}Fe₂O₄ were administered and an alternating current magnetic field was applied.

FIG. 6 shows the tumor size in an FSaLL mouse tumor model as a control group and an experimental group to which superparamagnetic nanoparticles Mn_{0.5}Zn_{0.5}Fe₂O₄ were administered and an alternating current magnetic field was applied, as observed through tissue staining.

FIG. 7 is the results of confirming the expression levels of Ki67, active caspase-3, and TUNEL in an experimental group to which superparamagnetic nanoparticles Mn_{0.5}Zn_{0.5}Fe₂O₄ were administered and an alternating current magnetic field was applied, compared to an FSaLL mouse tumor model as a control group.

FIG. 8 is the results of confirming the expression levels of CD31 (PECAM-1) and CD3 (T cell) in an experimental group to which superparamagnetic nanoparticles Mn_{0.5}Zn_{0.5}Fe₂O₄ were administered and an alternating current magnetic field was applied, compared to an FSaLL mouse tumor model as a control group.

FIG. 9 is the results of confirming the expression levels of CD4 and CD8 in an experimental group to which superparamagnetic nanoparticles Mno. 5Zn_{0.5}Fe₂O₄ were administered and an alternating current magnetic field was applied, compared to an FSaLL mouse tumor model as a control group.

FIG. 10 is the results of confirming the expression levels of CD161a and Ibal in an experimental group to which superparamagnetic nanoparticles Mn_{0.5}Zn_{0.5}Fe₂O₄ were administered and an alternating current magnetic field was applied, compared to an FSaLL mouse tumor model as a control group.

FIG. 11 is the results of confirming the expression levels of CD45RA, CD138, and Ly6C in an experimental group to which superparamagnetic nanoparticles Mn_{0.5}Zn_{0.5}Fe₂O₄ were administered and an alternating current magnetic field was applied, compared to an FSaLL mouse tumor model as a control group.

FIG. 12 is the results of confirming the expression levels of HSP60 and HSP70 in an experimental group to which superparamagnetic nanoparticles Mn_{0.5}Zn_{0.5}Fe₂O₄ were administered and an alternating current magnetic field was applied, compared to an FSaLL mouse tumor model as a control group.

FIG. 13 shows the change in tumor temperature over time after administration of superparamagnetic nanoparticles Mn_{0.5}Zn_{0.5}Fe₂O₄ and application of a magnetic field to a U87MG mouse tumor model.

FIG. 14 shows the change in tumor size in experimental groups (experimental group 1: application of AC magnetic field three times, experimental group 2: application of AC magnetic field six times) to which superparamagnetic nanoparticles Mn_{0.5}Zn_{0.5}Fe₂O₄ were administered and an alternating current magnetic field was applied, compared to an FSaLL mouse tumor model as a control group.

FIG. 15 shows brain MR images in a U87MG tumor model as a control group (control), a group to which Mn_{0.5}Zn_{0.5}Fe₂O₄ nanoparticles were administered to a U87MG tumor model (MNP group), and a group to which Mn_{0.5}Zn_{0.5}Fe₂O₄ nanoparticles were administered and a magnetic field was applied three times to a U87MG tumor model (MNP+AMF group).

FIG. 16 shows the change in tumor size in a U87MG tumor model as a control group and an experimental group to which superparamagnetic nanoparticles Mn_{0.5}Zn_{0.5}Fe₂O₄ were administered and an alternating current magnetic field was applied.

FIG. 17 is the results of confirming the expression levels of Ki67, active caspase-3, and TUNEL in a U87MG tumor model and an experimental group to which superparamagnetic nanoparticles Mn_{0.5}Zn_{0.5}Fe₂O₄ were administered and an alternating current magnetic field was applied.

FIG. 18 is the results of confirming the expression levels of CD45RA and CD138 in a U87MG tumor model as a control group and an experimental group to which superparamagnetic nanoparticles Mn_{0.5}Zn_{0.5}Fe₂O₄ were administered and an alternating current magnetic field was applied.

FIG. 19 is the results of confirming the expression levels of CD3 and Ly6C in a U87MG tumor model as a control group and an experimental group to which superparamagnetic nanoparticles Mn_{0.5}Zn_{0.5}Fe₂O₄ were administered and an alternating current magnetic field was applied.

FIG. 20 is the results of confirming the expression levels of CD4 and CD8 in a U87MG tumor model as a control group and an experimental group to which superparamagnetic nanoparticles Mn_{0.5}Zn_{0.5}Fe₂O₄ were administered and an alternating current magnetic field was applied.

FIG. 21 is the results of confirming the expression levels of CD161 and Ibal in a U87MG tumor model as a control group and an experimental group to which superparamagnetic nanoparticles Mn_{0.5}Zn_{0.5}Fe₂O₄ were administered and an alternating current magnetic field was applied.

FIG. 22 is the results of confirming the expression levels of HSP60 and HSP70 in a U87MG tumor model as a control group and an experimental group to which superparamagnetic nanoparticles Mn_{0.5}Zn_{0.5}Fe₂O₄ were administered and an alternating current magnetic field was applied.

FIGS. 23 to 25 are the results of observing, using fluorescence microscopy, the distribution of nanoparticles and immune cells on the surface of cancer tissue before and after administration of superparamagnetic nanoparticles Mn_{0.5}Zn_{0.5}Fe₂O₄ and application of an alternating current magnetic field to a U87MG tumor model (red: nanoparticles, green: fluorescent markers, blue: blood vessels, white: immune cells).

FIGS. 26 to 31 are the results of observing, using fluorescence microscopy, the distribution of nanoparticles and immune cells in cancer tissue before and after administration of superparamagnetic nanoparticles Mn_{0.5}Zn_{0.5}Fe₂O₄ and application of an alternating current magnetic field to a U87MG tumor model, (red: nanoparticles, green: fluorescent markers, blue: blood vessels, white: immune cells).

### BEST MODEL

Hereinafter, the composition and effects the present disclosure will be described in more detail with reference to Examples. These Examples are intended solely for the purpose of illustrating the present disclosure and are not intended to limit its scope.

### Example 1: Synthesis and Surface Modification of Nanoparticles

### 1-1. Preparation of Superparamagnetic Nanoparticles

Fe (III) acetylacetonate (Fe (acac)₃, Aldrich Chemical Co.), Mn (II) acetate tetrahydrate (Aldrich Chemical Co.), Zn (II) acetate dihydrate (Aldrich Chemical Co.) , oleic acid (Aldrich Chemical Co.) , oleylamine (Aldrich Chemical Co.) , benzyl ether (Aldrich Chemical Co.) , and 1,2-hexadecanediol (Tokyo Chemical Industries Co.) were mixed to synthesize solid-state MnₓZn₁₋ₓFe₂O₄ nanoparticles.

Specifically, Fe (III) acetylacetonate (2 mmol), Mn (II) acetate tetrahydrate (0.5 mmol), Zn (II) acetate dihydrate (0.5 mmol), and 1,2-hexadecanediol (10 mmol) were mixed in powder form in a three-necked round-bottom flask, followed by the addition of 2.4 mL of oleic acid and 3.5 ml of oleylamine (step 1). Then, after adding 20 mL of benzyl ether, the flask was fixed inside a heating mantle for a round-bottom flask and heated to 280 °C at a rate of 12 to 13 °C/min for 20 minutes, then cooled to 270 °C for 1 hour (step 2). Subsequently, it was heated to 296.5 °C at a rate of 2 to 3 °C/min for 10 minutes, and then maintained at that temperature for 46.5 minutes (step 3). The temperature was lowered to 80 °C, and then 40 ML of ethanol was added thereto to prepare Mn_{0.5}Zn_{0.5}Fe₂O₄ (step 4).

### 1-2. Surface Modification of Synthesized Superparamagnetic Nanoparticles

The synthesized nanoparticles were coated with methoxy-PEG-silane 500 Da (Dalton), which is a biocompatible polymer. To coat a PEG layer, the surface of the synthesized nanoparticles was first modified with oleic acid. Oleic acid (3 mL) and NH₄Cl (0.7 mL) were added to an ethanol solution along with the nanoparticles. The mixture was stirred vigorously for 2 hours, and then the nanoparticles were precipitated by a permanent magnet and washed with acetone to obtain oleic acid-coated nanoparticles. The oleic acid-coated nanoparticles were dispersed in toluene (7.5 mL), followed by the addition of triethylamine (3.75 mL) and methoxy-PEG-silane 500 Da (0.75 ml). The mixed solution was stirred well for 24 hours. The PEG-coated nanoparticles in the solution were washed with pentane and dispersed in water to produce a nanofluidic solution.

### Example 2: Characterization of Synthesized Superparamagnetic Nanoparticles

### 2-1. Comparison of Thermal Characteristics and Yields According to Preparation Methods

The thermal characteristics of Mn_{0.5}Zn_{0.5}Fe₂O₄ prepared by the method of Example 1 -1 were compared with those of Mn_{0.5}Zn_{0.5}Fe₂O₄ prepared by the conventional method. The conventional method differs from the preparation method of Example 1 -1 in that a hot plate is used instead of a heating mantle; in step 2, it is heated to 200 °C at a rate of 8 to 9 °C/min for 20 minutes and then maintained at that temperature for 1 hour; and in step 3, it is heated to 296.5 °C at a rate of 9 to 10 °C/min for 10 minutes.

The thermal characteristics and yields of Mn_{0.5}Zn_{0.5}Fe₂O₄ prepared by the method of Example 1 -1 and the conventional method, respectively, were compared.

Specifically, the thermal characteristics were measured using a specially designed AC magnetic field generation system consisting of an AC coil, a capacitor, a DC power source, a wave generator, and a PC system. The AC magnetic field generation system operates in a wide frequency range from 0 to 380 KHz, which has no harmful effects on the human body, with a magnetic field strength of up to 350 Oe. Since nanoparticles have different AC heating capacities depending on their material, particle size, and size distribution, the AC heating capacity of various nanoparticles should be evaluated over a wide range of frequencies and magnetic fields. The total amount of solid nanoparticles measured for AC heating characteristics was fixed at 60 mg in an Eppendorf-tube. To design the measurement environment as an isolated system (to avoid interference of AC heating measurements from the surrounding atmosphere), each sample was placed on an insulating Styrofoam in the center of a sample bed. The tip of the optical fiber was placed inside an Eppendorf tube containing solid-state Mn_{0.5}Zn_{0.5}Fe₂O₄ nanoparticles. Mn_{0.5}Zn_{0.5}Fe₂O₄ nanoparticles prepared by the method of Example 1 -1 and the conventional method, respectively, were investigated at eight different frequencies (31.9, 47.0, 98.9, 140.0, 168.1, 195.5, 239.9, and 360.2 kHz) and five different magnetic field intensities (80, 100, 120, 140, and 160 Oe). A total time of magnetic field generation was 600 s for each measurement. When the magnetic field was turned on, the AC heating temperature was measured with an optical thermometer, and cooling was performed when the magnetic field was turned off.

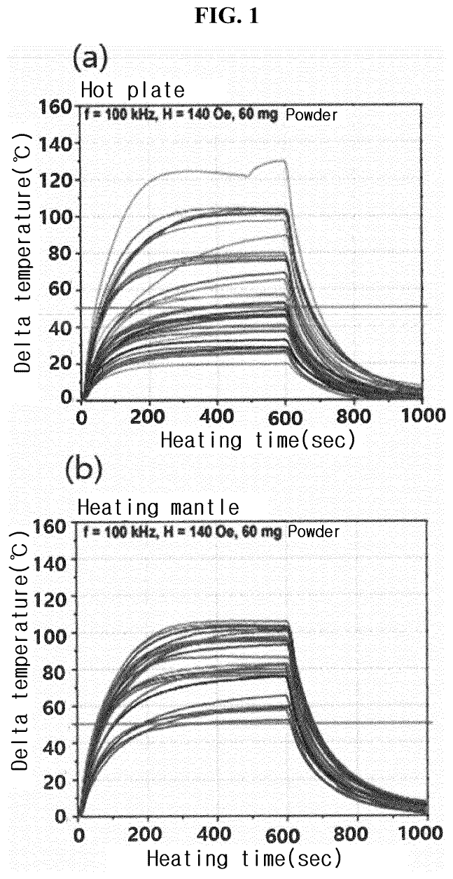

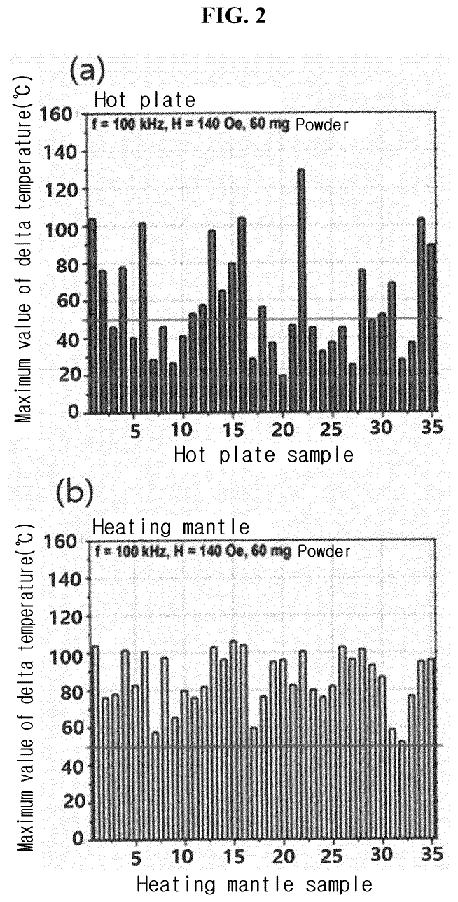

As a result, it was confirmed that Mn_{0.5}Zn_{0.5}Fe₂O₄ prepared by the method of Example 1 -1 exhibited stable thermal characteristics compared to Mn_{0.5}Zn_{0.5}Fe₂O₄ prepared by the conventional method (FIG. 1, a: conventional method, b: method of Example 1-1), and all samples exhibited thermal characteristics of 50 °C or more, SO that excellent superparamagnetic nanoparticles were prepared at a yield of 100%. In addition, it was confirmed that superparamagnetic nanoparticles exhibiting thermal characteristics of 50 °C or more were prepared at a yield of 49% when prepared by the conventional method (FIG. 2, a: conventional method, b: method of Example 1-1).

### 2-2. Measurement of Magnetically-Induced Heating Characteristics

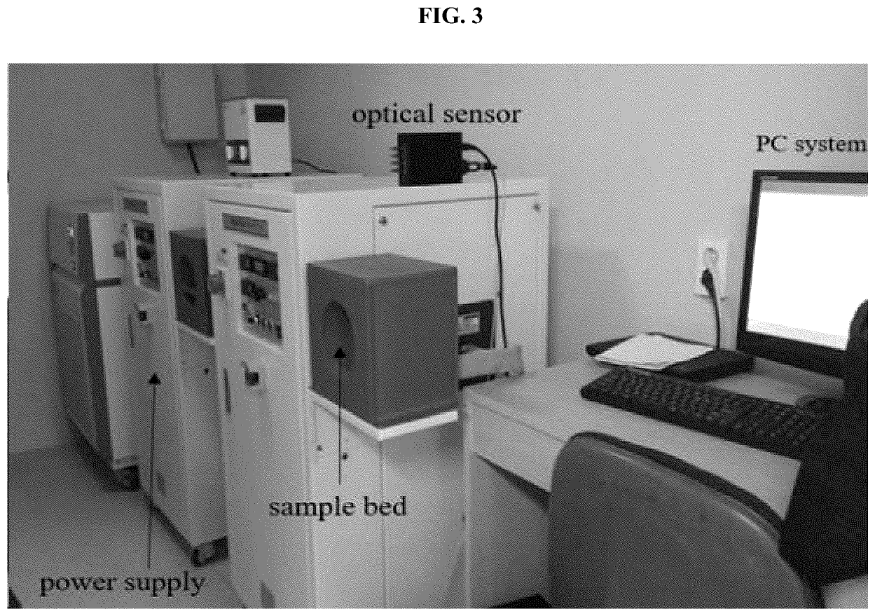

The heating characteristics induced by an AC magnetic field of Mn_{0.5}Zn_{0.5}Fe₂O₄ nanoparticles prepared by the method of Example 1 -1 were measured using a specially designed AC magnetic field generation system consisting of an AC coil, a capacitor, a DC power supply, a wave generator, and a PC system (FIG. 3).

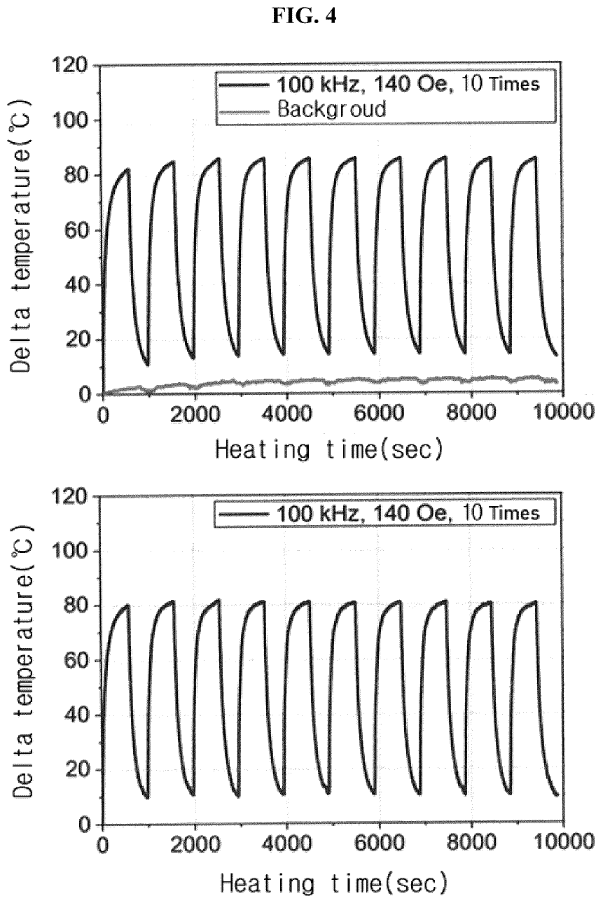

It was confirmed that repeated exposure of 60 mg of Mn_{0.5}Zn_{0.5}Fe₂O₄ with a size of 7 nm to an alternating current magnetic field with a frequency of 100 kHz and an intensity of 140 Oe for ten times resulted in uniform heat generation up to 80 °C (FIG. 4).

### Example 3: Confirmation of Cancer Immunotherapy Efficacy of Synthesized Superparamagnetic Nanoparticles

### 3-1. Mouse Tumor Model

Animal treatment, subcutaneous fibrosarcoma cell injection, glioblastoma injection, and in vivo hyperthermia experiments were performed according to the guidelines approved by the Institutional Animal Care and Use Committee (IACUC) of Seoul National University Hospital (IACUC No. SNU-161129-2). All procedures were performed to reduce the amount of pain caused to Balb/c nude mice. For a fibrosarcoma mouse tumor model, the mice used in this experiment were anaesthetized and FSaLL cells (radiation-induced fibrosarcoma of C3H mice, created in the laboratory of Dr. Herman Suit laboratory at Massachusetts General Hospital; early generation cells were donated by Dr. Suit) (5×10⁶ cells/50 μL) were administered by subcutaneous injection into the proximal femoral region of Balb/c nude mice (FSaLL mouse tumor model).

For a glioblastoma model, 5×10⁵ cells/5 μL of glioblastoma cells (U87MG) were administered by injection into the brain of nude mice (U87MG mouse tumor model).

### 3-2. Analysis of Immune Cell Increase

The cancer immunotherapy efficacy of synthesized superparamagnetic nanoparticles was confirmed in each mouse model.

First, the FSaLL mouse tumor model was anesthetized and 100 μL (30 mg/ml) of PEG-coated Mn_{0.5}Zn_{0.5}Fe₂O₄ nanoparticles were injected into the center of the induced tumor. The mice were placed in the center of an AC coil system for magnetic hyperthermia and subjected to a magnetic field with an intensity of 140 Oe and a frequency of 100 kHz. For hyperthermia therapy, the AC magnetic field was applied six times for 20 minutes each time.

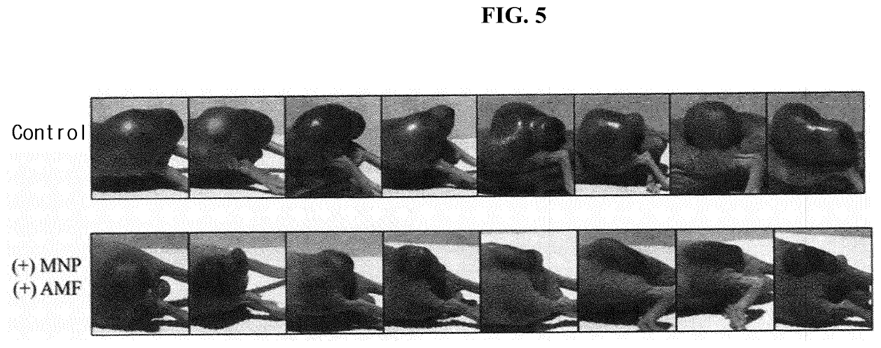

As a result, it was confirmed with the naked eye that the tumor size decreased over time in the experimental group to which superparamagnetic nanoparticles were administered and an AC magnetic field was applied, compared to the FSaLL mouse tumor model as the control group (FIG. 5).

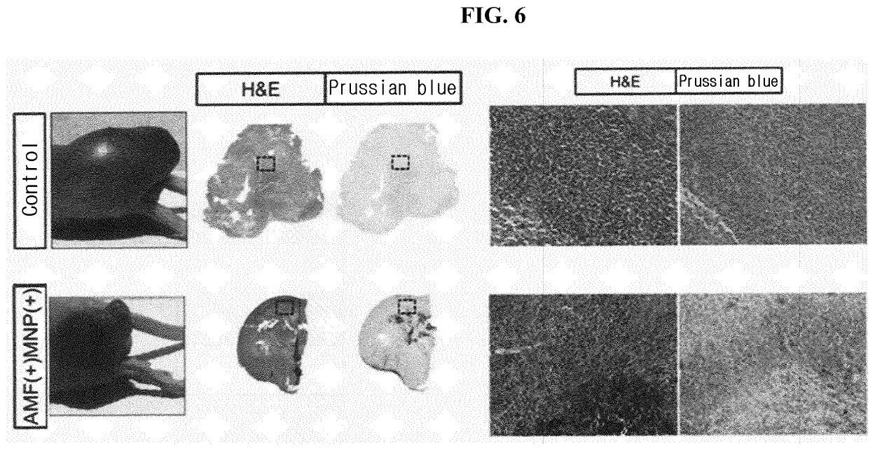

Further, it was confirmed that the tumor size significantly decreased in tissue staining using H&E staining and Prussian Blue (FIG. 6). H&E and Prussian Blue staining are widely used in medical diagnostics. The tissue suspected of being cancerous can be biopsied through pathology and tissue sections can be stained with H&E and Prussian Blue staining to confirm the composition of the corresponding tissue.

In addition, changes in the expression of Ki67, active caspase-3, and TUNEL were confirmed through immunohistochemistry. By confirming the degree of distribution of nuclear proteins in tumor cells through Ki67, it is possible to determine how actively cell division is occurring in the tumor cells. In contrast, active caspase-3 and TUNEL, which are markers for cell death, indicate the progression of a suicidal mechanism in which cells die on their own, called apoptosis. Therefore, the more such markers are activated and displayed, the more they can be interpreted as indicating a decrease in tumor cells.

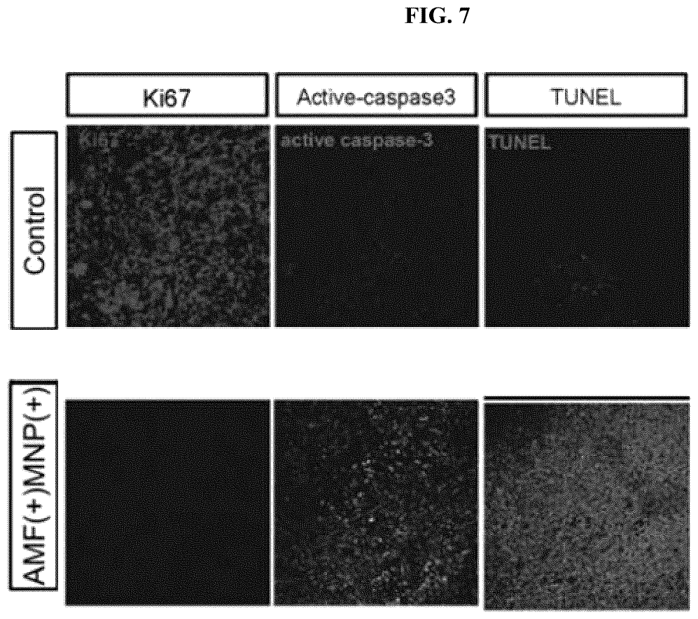

It was confirmed from the experimental results that the expression of Ki67 decreased and the expression of active caspase-3 and TUNEL increased in the experimental group compared to the control group (FIG. 7).

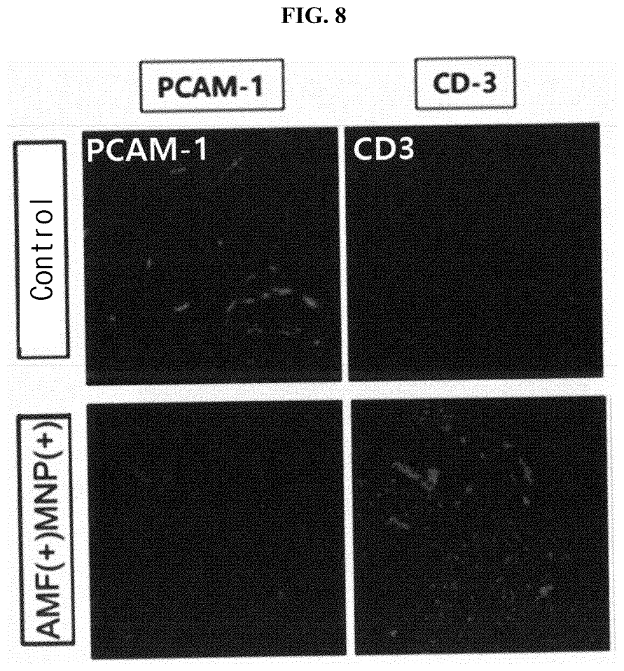

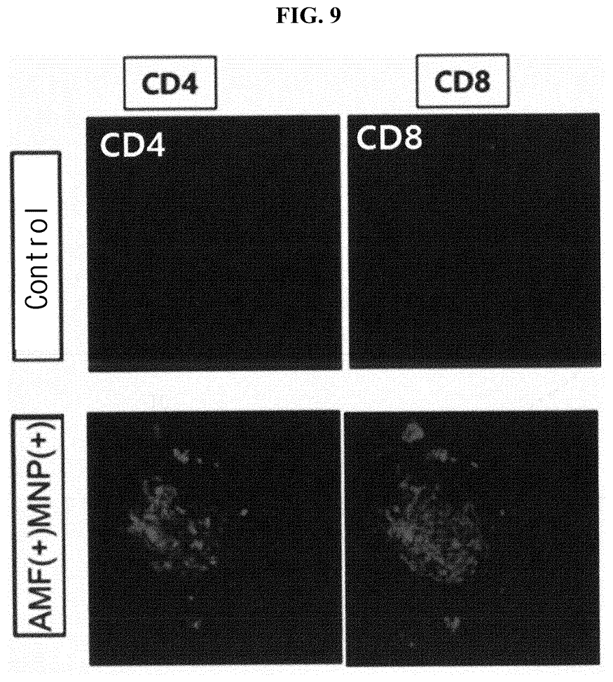

Furthermore, it was confirmed that the expression of CD31 (PECAM-1) decreased, but the expression of CD3 (T cell) increased significantly (FIG. 8), and the expression of CD4 and CD8 increased in the experimental group compared to the control group (FIG. 9).

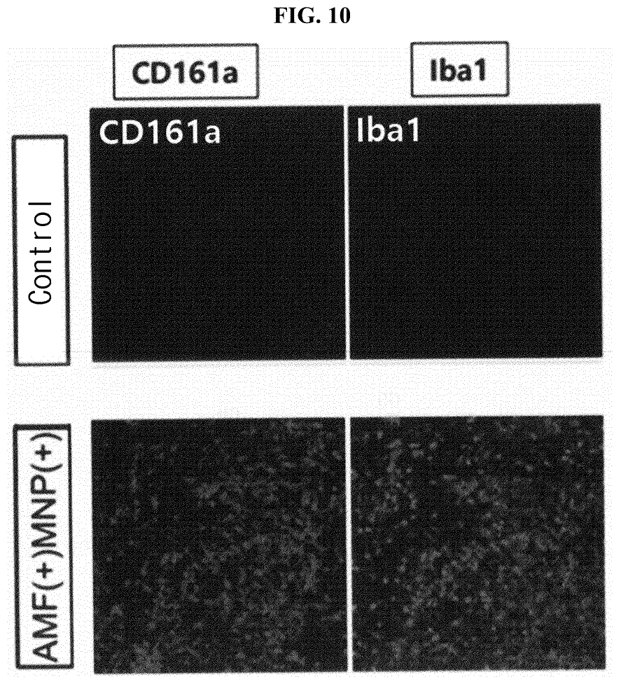

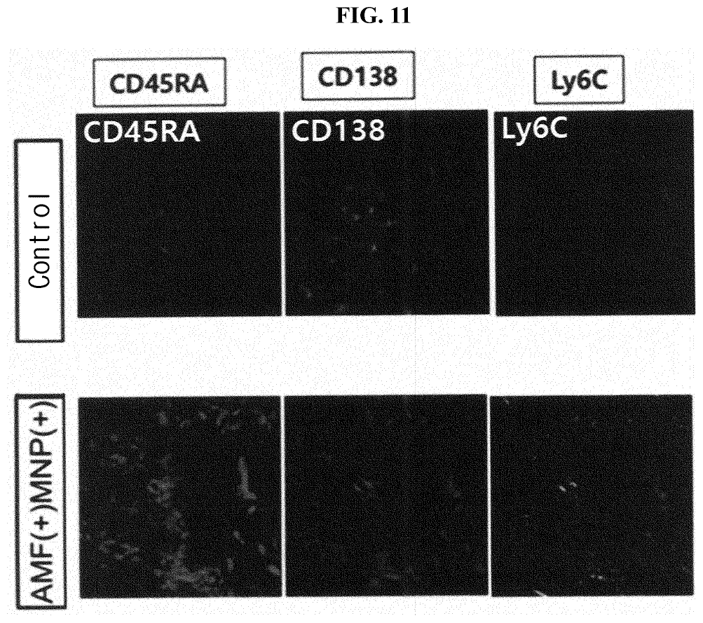

In addition, it was confirmed that CD161a, which is a natural killer cell (NK cell) maker, and Ibal, which is a macrophage maker, increased significantly (FIG. 10), and CD45RA (naive T cell, B cell), CD138 (plasma cell), and Ly6C, which is a macrophage maker, increased significantly in the experimental group compared to the control group (FIG. 11), confirming that immune cells increased in the experimental group to which superparamagnetic nanoparticles were administered.

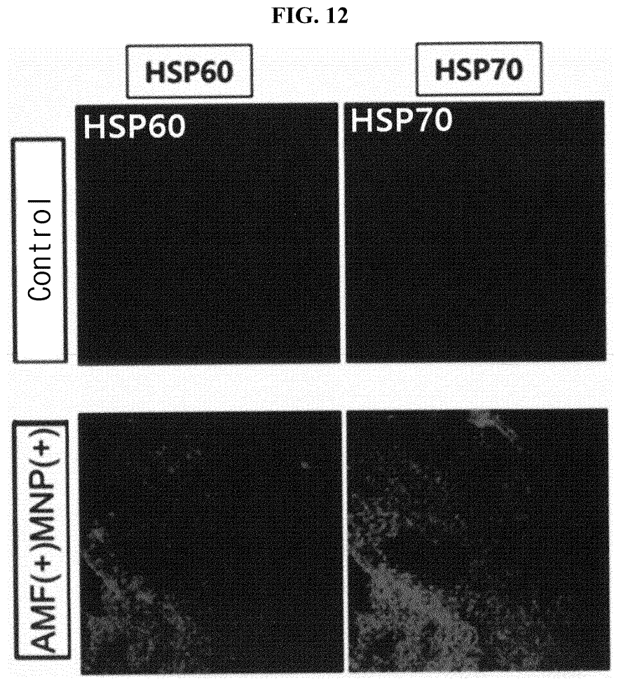

Also, it was confirmed that the expression of HSP60 and HSP70 increased in the experimental group compared to the control group, through immunohistochemical staining (FIG. 12). Heat shock protein (HSP) 60 and heat shock protein (HSP) 70 are representative HSPs and are well known to activate antigen presentation through the activation of immune cells such as macrophages and lymphocytes, and the activation and maturation of dendrite cells.

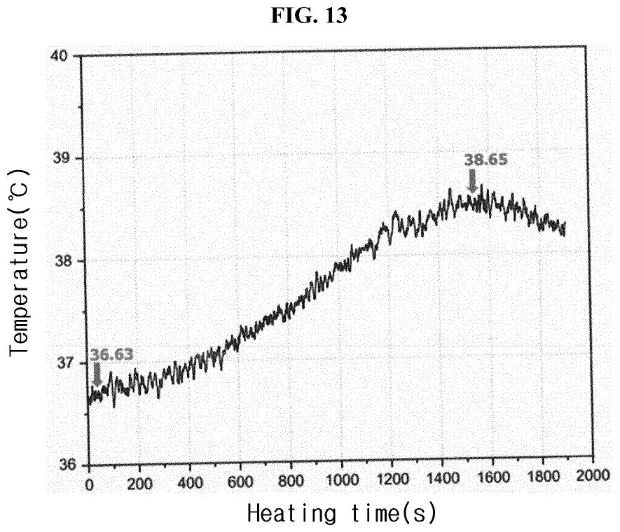

Subsequently, the U87MG mouse tumor model was anesthetized and 5 μL (30 mg/ml) of Mn_{0.5}Zn_{0.5}Fe₂O₄ nanoparticles were injected into the brain of the mouse. The mice were placed in the center of an AC coil system for magnetic hyperthermia and subjected to a magnetic field with an intensity of 140 Oe and a frequency of 100 kHz. For hyperthermia therapy, the AC magnetic field was applied six times for 20 minutes each time, and the temperature change of the tumor during the application of the magnetic field increased from an initial 36.63 °C up to 38.65 °C and then decreased, as shown in FIG. 13.

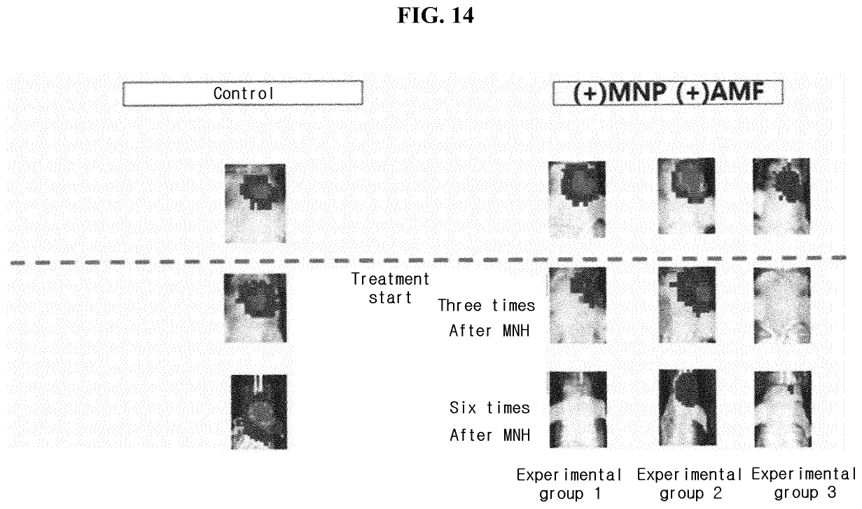

From the IVIS image results obtained by staining with GFP and Luciferase in the U87MG tumor model as the control group (control) and experimental groups 1 and 2 to which 5 μl of Mn_{0.5}Zn_{0.5}Fe₂O₄ nanoparticles were administered and magnetic hyperthermia (MHT) was performed by applying an AC magnetic field three or six times to the U87MG tumor model, it was confirmed that the tumor size in the control group increased significantly, while the tumor size in experimental groups 1 and 2 decreased (FIG. 14).

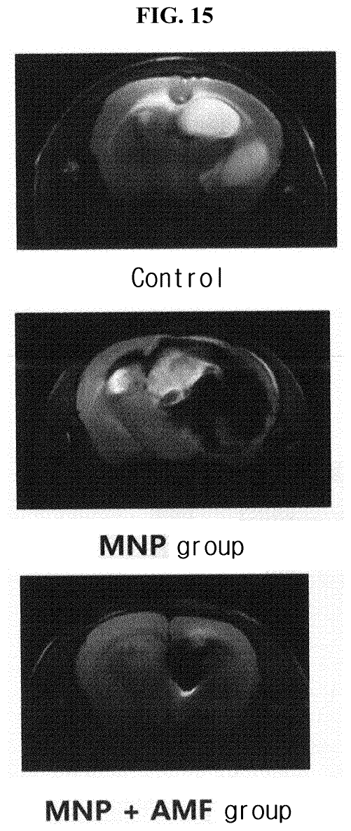

Also, the brain MR images (scanning conditions: TR: 2000 ms/TE: 48 ms, FA: 90, 1.0 mm thickness, spin echo) taken after treating the U87MG tumor model as the control group (control), the group to which Mn_{0.5}Zn_{0.5}Fe₂O₄ nanoparticles were administered to the U87MG tumor model (MNP group), and the group to which Mn_{0.5}Zn_{0.5}Fe₂O₄ nanoparticles were administered and a magnetic field was applied three times to the U87MG tumor model (MNP+AMF group), with GFP and Luciferase, are shown in FIG. 15.

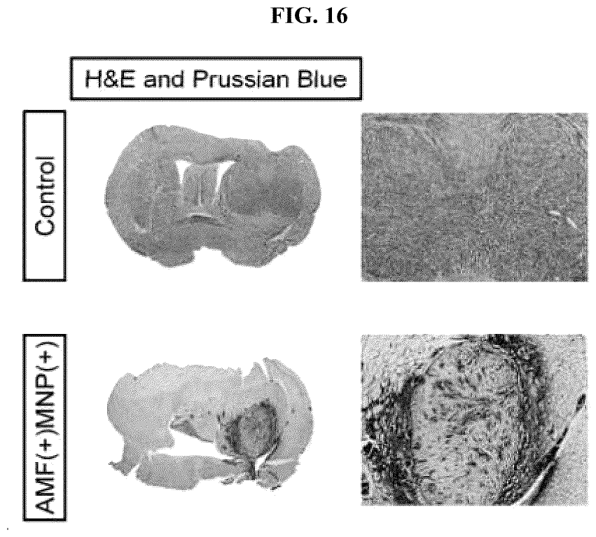

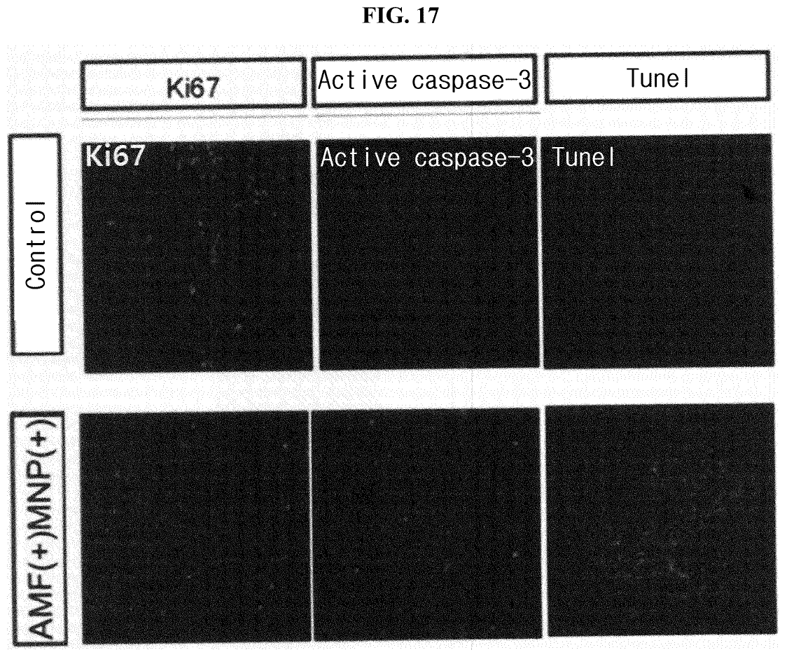

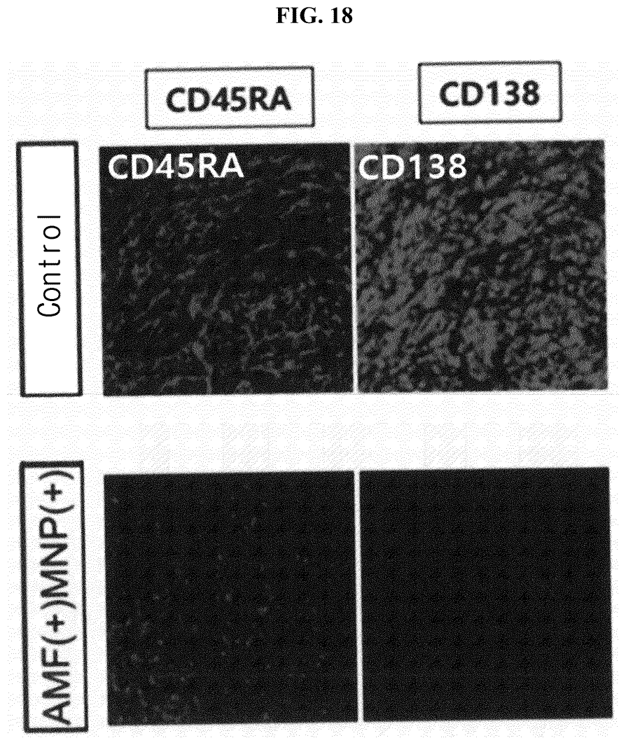

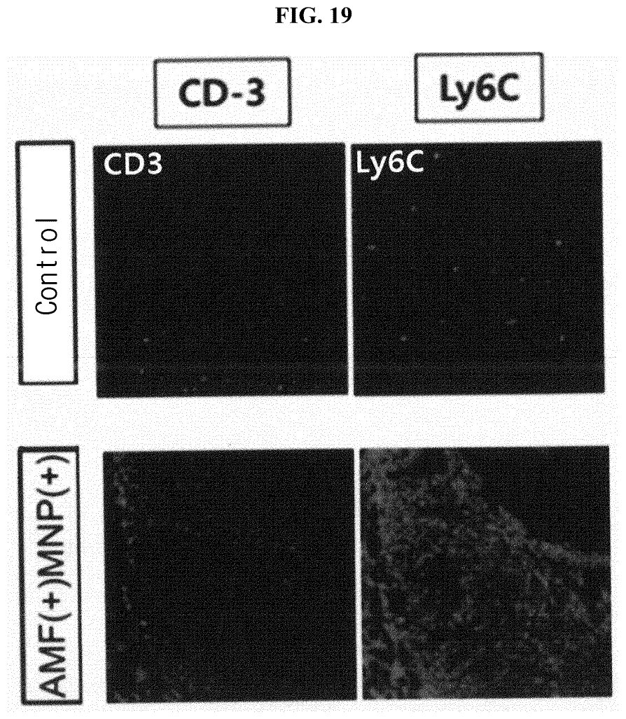

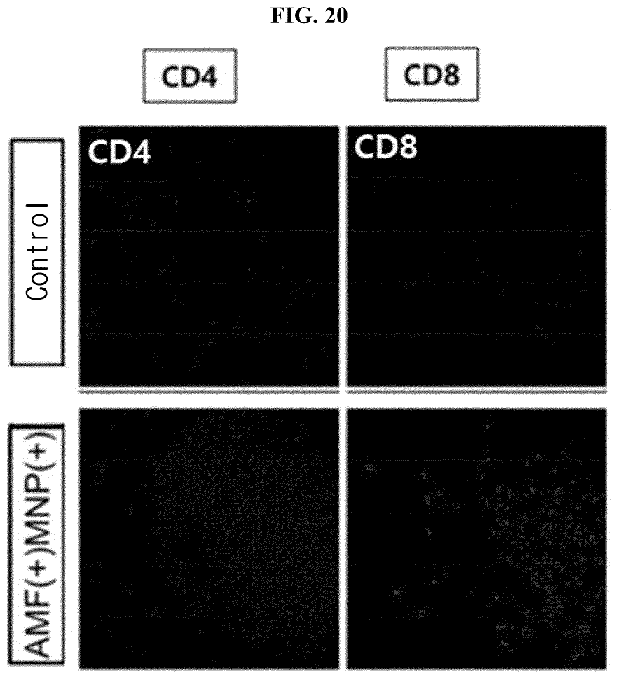

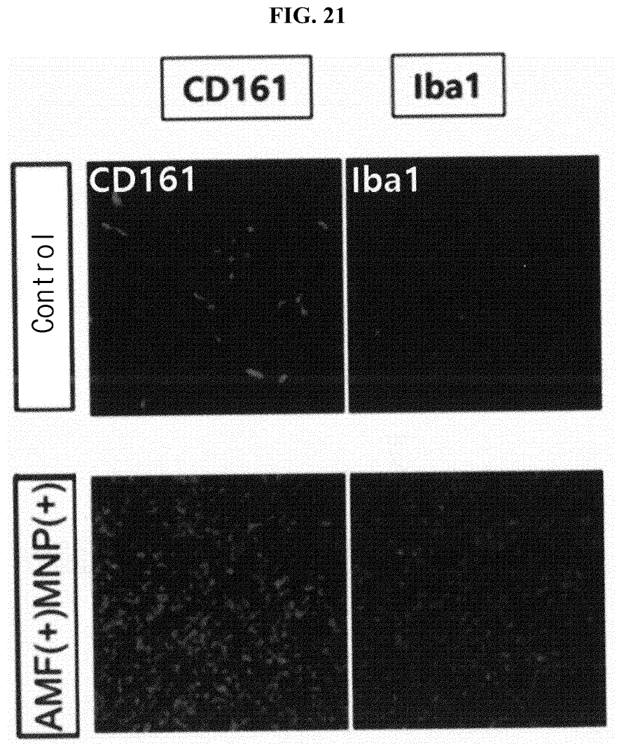

It was confirmed that the tumor size decreased significantly in the experimental group to which superparamagnetic nanoparticles were administered and an alternating current magnetic field was applied, compared to the U87MG tumor model as the control group (FIG. 16). As a result of the immunohistochemical test, it was confirmed that the expression of Ki 67 decreased and the expression of active caspase-3 and TUNEL increased in the experimental group compared to the control group (FIG. 17). Similarly, it was confirmed that CD45RA (B cell, naive T cell) and CD138 (plasma cell) increased significantly in the experimental group compared to the control group (FIG. 18). Furthermore, it was confirmed that CD3 (T cell) and Ly6C, which is a macrophage maker, increased in the experimental group compared to the control group (FIG. 19), and that CD4 (helper T cell) and CD8 (cytotoxic T cell) increased (FIG. 20), and CD161, which is a natural killer cell (NK cell) marker and Ibal, which is a microglia marker, increased (FIG. 21).

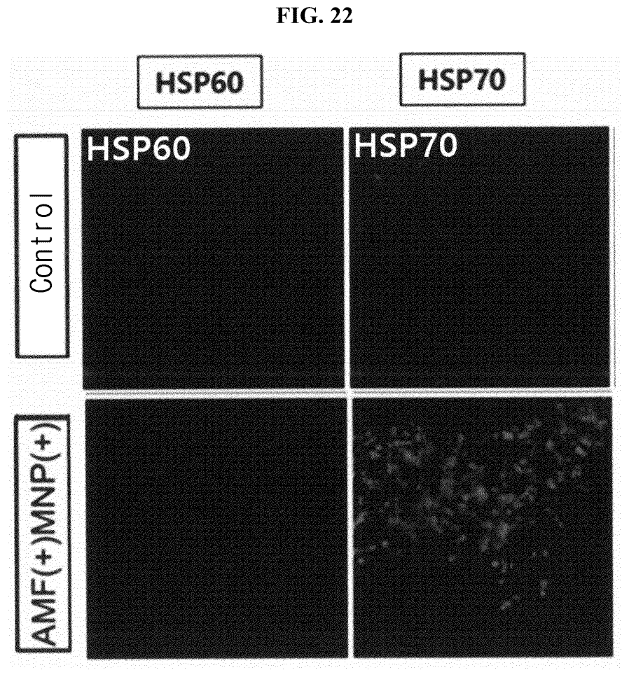

Furthermore, it was confirmed that the expression of HSP60 and HSP70 increased in the experimental group compared to the control group (FIG. 22).

Thus, from the above results, it was confirmed that Mn_{0.5}Zn_{0.5}Fe₂O₄ nanoparticles have immunotherapeutic efficacy by inducing an increase in immune cells such as macrophages, natural killer cells, and T cells.

3-3. Analysis of Distribution of Superparamagnetic Nanoparticles in Cancer Tissues of Animal Models After administering Mn_{0.5}Zn_{0.5}Fe₂O₄ nanoparticles to the U87MG tumor model, the distribution of nanoparticles and immune cells in cancer tissues before and after applying an alternating current magnetic field was observed using fluorescence microscopy.

Specifically, tumor cells of U87MG tumor model and Mn_{0.5}Zn_{0.5}Fe₂O₄ nanoparticles and blood vessels were fluorescently labeled, and then 10 mg/kg of Mn_{0.5}Zn_{0.5}Fe₂O₄ nanoparticles were administered by intra-arterial injection into the carotid artery of a mouse model and imaged by IVM-CM (Fontocal & Two-photon convertible microscopy) at 1-day intervals for a total of 4 days starting from the day after administration. The same location was tracked and imaged at each time point, and the excitation laser source (Ex) and emission spectra (Em) for each fluorescent marker were as follows. Blood vessels (fluorescent marker: CD31-mFluor 405) Ex. 405, Em. 425-465; tumors (fluorescent marker: U 87MG-GFP) Ex. 488, Em. 500-550; Mn_{0.5}Zn_{0.5}Fe₂O₄ nanoparticles Ex. 640, Em. 663-733.

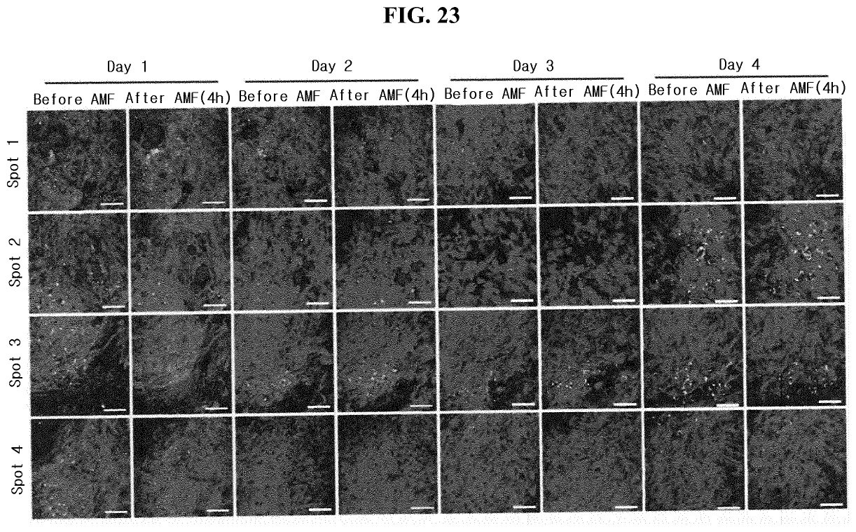

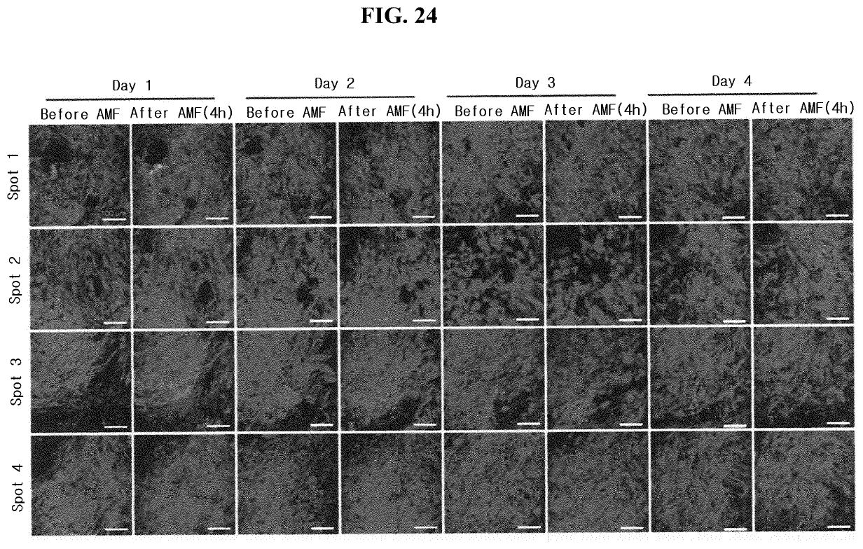

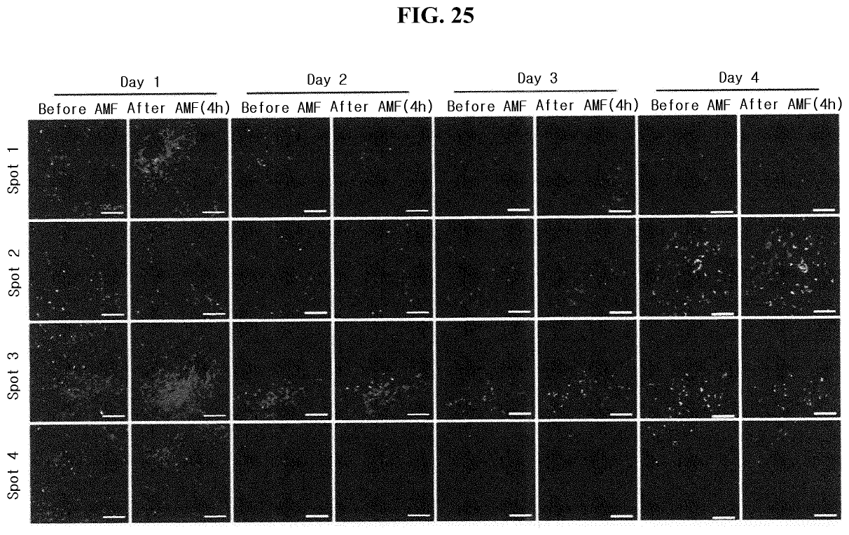

As a result, in the case of the surface of the cancer tissue, as shown in FIGS. 23 to 25, at the beginning (day 1 to day 2), most of the nanoparticles were located in the empty space between cancer cells or around blood vessels, and as time passed, a large amount of nanoparticles were observed in the form being captured by immune cells. In addition, at day 1 to day 2, the observed nanoparticles increased after the application of the alternating current magnetic field, and at day 3 to day 4, there was no significant difference in the distribution of nanoparticles between the time points before and after the application of the AC magnetic field (red: nanoparticles, green: fluorescent markers, blue: blood vessels, white: immune cells).

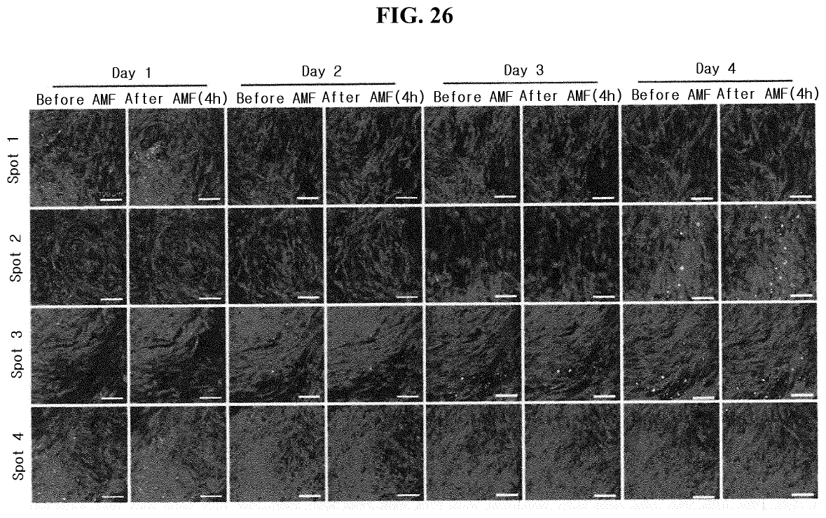

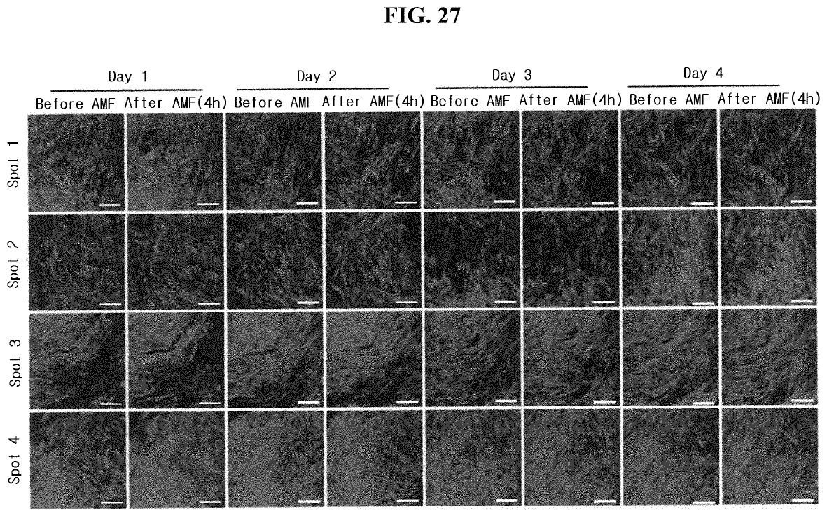

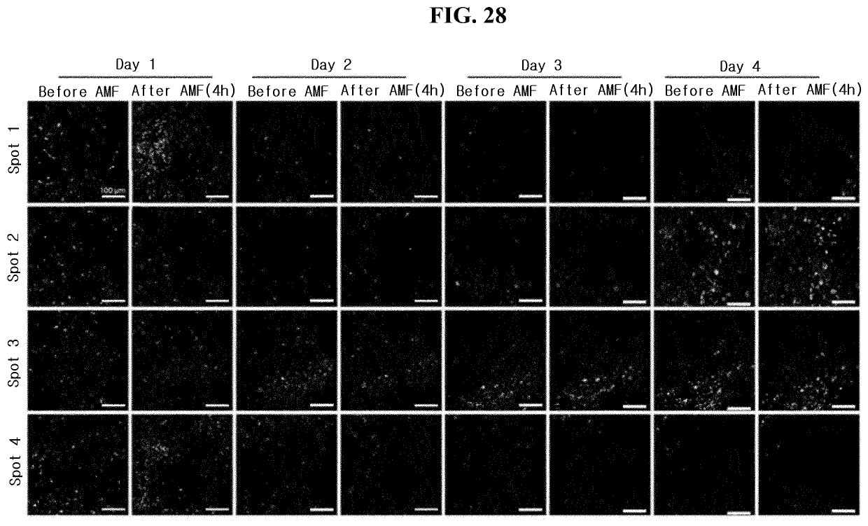

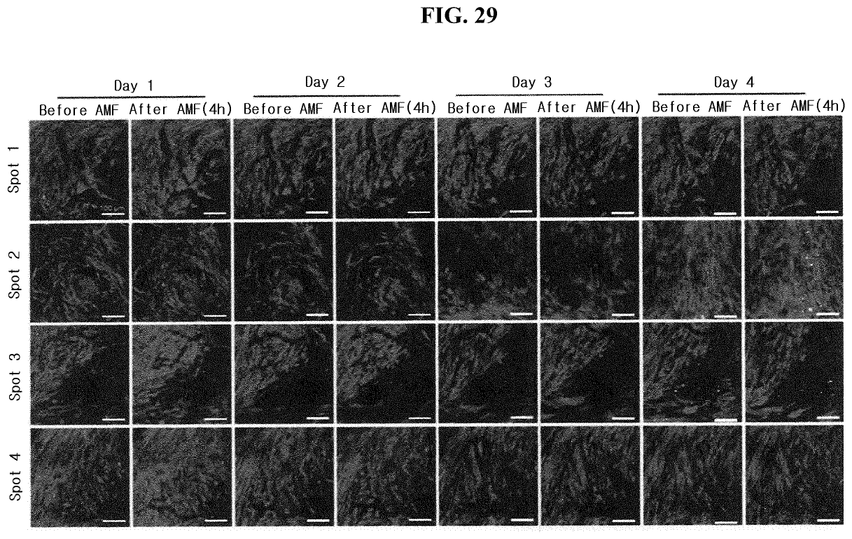

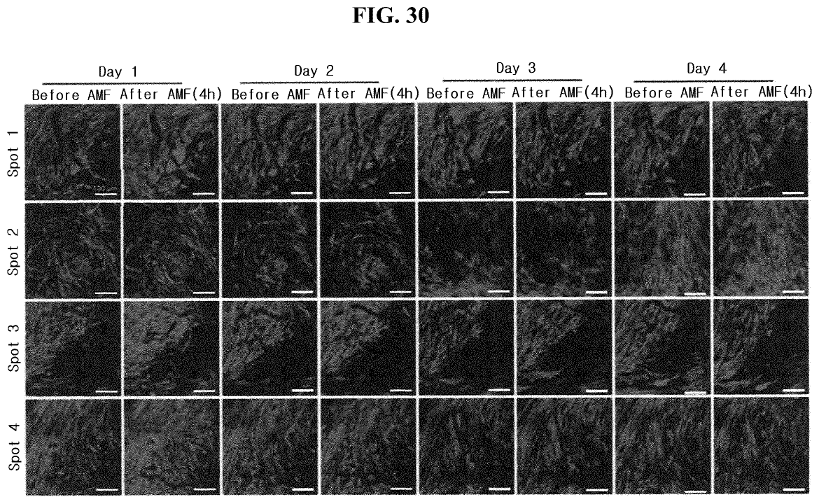

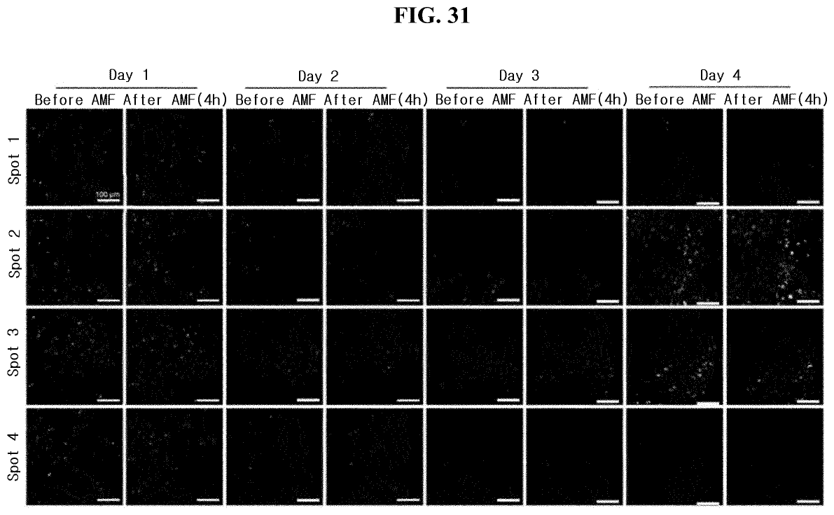

In the case of the cancer tissue, as shown in FIGS. 26 to 31, most of the nanoparticles were observed in the empty spaces between cancer cells or in the form being captured in immune cells distinguished by fluorescent signals, with some locations showing a decrease in nanoparticle coverage over time and then an increase again.

### These Results Confirmed That Administration of Mn_{0.5}Zn_{0.5}Fe₂O₄ nanoparticles to cancer cells in vivo induced an increase in immune cells.

From the above description, those skilled in the art to which the present disclosure pertains will understand that the present disclosure can be implemented in other specific forms without changing its technical idea or essential features. In this regard, it should be understood that the embodiments described above are exemplary in all respects and are not intended to be limiting. The scope of the present disclosure should be interpreted that all changes or modifications derived from the meaning and scope of patent claims to be described below rather than the detailed description above and their equivalent concepts are included within the scope of the present disclosure.

1. A method for cancer immunotherapy or for ameliorating, treating, or preventing a decrease in immune responses in a subject with cancer, comprising: administering to the subject a composition comprising_a superparamagnetic nanoparticle represented by the following Formula 1 as an active ingredient:
[Formula 1]
KₓL_{y}Fe₂₍₃₎O₄ (0≤x≤3, 0≤y≤3)
wherein K or L is a monovalent to trivalent cation metal including lithium (Li), sodium (Na), magnesium (Mg), calcium (Ca), manganese (Mn), zinc (Zn), aluminum (Al), or gallium (Ga).

2. The method claim 1, wherein the cancer immunotherapy or the amelioration, treatment, or prevention of a decrease in immune responses is achieved by an increase in immune cells.

3. The method of claim 2, wherein the immune cells are one or more selected from the group consisting of macrophages, natural killer cells, and T cells.

4. A method for treating an immune disease or enhancing immunity in a subject, comprising: administering to the subject a composition comprising_a superparamagnetic nanoparticle represented by the following Formula 1 as an active ingredient:
[Formula 1]
KₓL_{y}Fe₂₍₃₎O₄ (0≤x≤3, 0≤y≤3)
wherein K or L is a monovalent to trivalent cation metal including lithium (Li), sodium (Na), magnesium (Mg), calcium (Ca), manganese (Mn), zinc (Zn), aluminum (Al), or gallium (Ga).

5. The method of claim 4, wherein the treatment of the immune disease or the immune enhancement is achieved by an increase in immune cells.

6. The method of claim 5, wherein the immune cells are one or more selected from the group consisting of macrophages, natural killer cells, and T cells.

7. The method of claim 4, wherein the immune disease is an immunodeficiency disease, an infectious disease, or an autoimmune disease.

8.-10 (canceled)

11. A method for suppressing a decrease in immune function or enhancing immune function, administered in combination with a chemotherapeutic anticancer drug, or suppressing a decrease in immune function, caused by anticancer treatment or enhancing immune function in a subject, comprising: administering to the subject a composition comprising_a superparamagnetic nanoparticle represented by the following Formula 1 as an active ingredient:
[Formula 1]
KₓL_{y}Fe₂₍₃₎O₄ (0≤x≤3, 0≤y≤3)
wherein K or L is a monovalent to trivalent cation metal including lithium (Li), sodium (Na), magnesium (Mg), calcium (Ca), manganese (Mn), zinc (Zn), aluminum (Al), or gallium (Ga).

12. The method of claim 11, wherein the suppression of a decrease in immune function or the enhancement of immune function is achieved by an increase in immune cells.

13. The method of claim 12, wherein the immune cells are one or more selected from the group consisting of macrophages, natural killer cells, and T cells.

14. -40. (canceled)
